(19) United States
(12) Patent Application Publication
(10) Pub. No.: US 2001/0007036 A1
(43) Pub. Date: Jul. 5, 2001

(54) PROCESS FOR THE PRODUCTION OF STANOLS

(76) Inventors: JOERG SCHWARZER, HILDEN (DE); EGBERT GRITZ, DUESSELDORF (DE); BERNHARD GUTSCHE, HILDEN (DE); WERNER KRAUSE, DUESSELDORF (DE); STEPHEN W. TURNER, HAMILTON, OH (US)

Correspondence Address: COGNIS CORPORATION, 2500 RENAISSANCE BLVD., SUITE 200, GULPH MILLS, PA, 19406

(21) Appl. No.: 09/273,518

(22) Filed: Mar. 22, 1999

Related U.S. Application Data
(60) Provisional application No. 60/079,001, filed on Mar. 23, 1998.

(51) Int. Cl. C07J 9/00

(52) U.S. Cl. 552/545; 552/544

(57) ABSTRACT

Sterols are hydrogenated to stanols in the presence of hydrogenation catalysts in reduced amounts of organic solvent. Both alcohols and paraffin hydrocarbons and also mixtures of alcohols and paraffin hydrocarbons may be used as the solvent. By virtue of the reduced consumption of solvent and the high yield of hydrogenated sterol, the process is particularly suitable for the industrial production of stanols.

### CROSS-REFERENCE TO RELATED APPLICATIONS

This application claims priority from U.S. provisional application Serial No. 60/079,001, filed Mar. 23, 1998, the disclosure of which is incorporated herein by reference.

### STATEMENT REGARDING FEDERALLY SPONSORED RESEARCH OR DEVELOPMENT

Not Applicable.

### BACKGROUND OF THE INVENTION

This invention relates to a process for the production of stanols by solvent hydrogenation of sterols in the presence of a hydrogenation catalyst.

Stanols and stanol esters have hypocholesterolemic properties and, for this reason, are added to foods to lower the content of serum cholesterol. Stanols are obtained by hydrogenation of animal or vegetable sterols. One process for the production of an animal sterol is described in Augustine and Reardon, Organic Prep. and Proc. 1 (2), pp 107-109, 1969. In this process, 1 g of cholesterol in 100 ml of ethanol is used for hydrogenation on the palladium catalyst, the hydrogenation reaction being carried out over a period of up to 10 hours at room temperature.

The known process for the hydrogenation of sterols is characterized by the necessity to use large quantities of organic solvent (ratio by weight of sterol to solvent 1:100). This is undesirable not only because of the economic disadvantages, but also in terms of operational reliability and potential ecological damage. These limitations restrict the industrial usefulness of the known process. Accordingly, the problem addressed by the present invention was to develop a process in which the consumption of organic solvent could be drastically reduced and a high yield of hydrogenated sterols could be obtained.

In the hydrogenation of sterols, it is crucially important that the double bond in the ring system is saturated and, at the same time, the free OH group in the 3 position remains intact. This free OH group serves as a functional group in the further derivatization of the stanols. Accordingly, characteristic parameters for the hydrogenation are the iodine value as a measure of the degree of unsaturation of a compound and the hydroxyl value (OH value) which is a measure of the free OH groups. Ideally, therefore, there is no change in the OH value before and after hydrogenation. The yield of hydrogenated sterols can be monitored through the reduction in the iodine value. Accordingly, another problem addressed by the invention was to provide stanols having a low iodine value. At the same time, the hydroxyl value would be reduced as little as possible by the process.

### SUMMARY OF THE INVENTION

The present invention relates to a process for the production of stanols which comprises contacting a sterol with hydrogen in the presence of a catalyst-effective amount of a hydrogenation catalyst and an organic solvent wherein the ratio by weight of the sterol to the solvent is from about 1:2 to about 1:10. The process requires reduced amounts of solvent thereby obviating the need for an expensive and time-consuming alcohol removal operation. The process produces stanols having substantially complete reduction of the double bond between carbon atoms 5 and 6 as reflected in the low iodine values and substantially complete retention of the alcohol functionality at carbon 3 as evidenced by little or no difference in the hydroxyl numbers between the starting sterols and the product stanols.

### BRIEF DESCRIPTION OF THE SEVERAL VIEWS OF THE DRAWING

Not Applicable.

### DETAILED DESCRIPTION OF THE INVENTION

Other then in the operating examples, or where otherwise indicated, all numbers expressing quantities of ingredients or reaction conditions used herein are to be understood as being modified in all instances by the term “about”. Practice within the numerical limits stated is generally preferred. Also, throughout this description, unless expressly stated to the contrary: percent, “parts” of, and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description or of generation in situ by chemical reactions specified in the description, and does not necessarily preclude other chemical interactions among the constituents of a mixture once mixed.

The present invention relates to a process for the production of stanols in which sterols are hydrogenated in the presence of a hydrogenation catalyst in an organic solvent, wherein the ratio by weight of sterol to solvent being from about 1:2 to about 1:10.

It has surprisingly been found that the consumption of solvents can be reduced by more than about 90% compared with known processes and that, at the same time, a high yield of hydrogenated sterols which give satisfactory results in regard to iodine value and hydroxyl value can be obtained. Accordingly, the invention provides an industrially workable and ecologically more favorable process for the hydrogenation of sterols.

Sterols are a group of compounds based on the structure of (partly) hydrogenated cyclopenta[a]phenanthrene. Most sterols are tetracyclic and typically contain 27 to 30 carbon atoms. These compounds are characterized by the hydroxy group at carbon atom number 3 and by the absence of functional groups. In addition, sterols generally contain a double bond in the 5/6 position and, occasionally, even in the 7/8, 8/9 etc. positions. Depending on their provenance, sterols are divided into zoosterols (animal origin), phytosterols (vegetable origin) and mycosterols (formed by yeasts and fungi). The most important representative of the zoosterols is cholesterol while typical representatives of the phytosterols are ergosterols, campesterols, stigmasterols, brassicasterols and sitosterols. The hydrogenated analogs of the sterols, also known as stanols, do not occur in nature. One preferred embodiment of the present invention is characterized by the use of phytosterols obtained, for example, in the working up of soya, rapeseed and sunflower oil for the hydrogenation reaction. The use of sitosterols, especially β-sitosterol, is particularly preferred for the intended application. It is common to obtain sterols as mixtures of compounds such as, for example, GENEROL® 122N sterols, a trademark product of Henkel Corporation, Gulph Mills, Pa. GENEROL® 122N sterol contains 25-30% campesterol, 17-22% stigmasterol and, 45-50% sitosterol.

The process according to the invention may be carried out in a solvent such as an alcohol of the formula (I):

R¹—OH

wherein R¹ is an aliphatic, linear or branched hydrocarbon radical containing 1 to about 22 carbon atoms. Alcohols containing a C₄₋₁₂ hydrocarbon radical are preferred. Typical examples are methanol, ethanol, n-propanol, i-propanol, n-butanol, i-butanol, sec.butanol, tert.butanol, pentanol, caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Butanol is particularly preferred. The process according to the invention can also be carried out in aliphatic linear or branched paraffin hydrocarbons having from about 5 to about 16 carbon atoms which are present in liquid form under the reaction conditions. Paraffin hydrocarbons containing a C₆₋₁₂ hydrocarbon radical such as, for example, hexane, heptane, octane, nonane, decane, undecane and dodecane and isomers thereof or isomer mixtures are preferably used. In another embodiment, the sterols are hydrogenated in a mixture of alcohols corresponding to formula (I) and aliphatic, linear or branched paraffin hydrocarbons containing 5 to 16 carbon atoms. Alcohols containing a C₁₋₁₂ hydrocarbon radical and aliphatic, linear or branched paraffin hydrocarbons containing a C₆₋₁₂ hydrocarbon radical are preferably used. In one preferred embodiment, the hydrogenation is carried out in a mixture of heptane and methanol. The ratio by weight of sterols to solvent is typically from about 1:2 to about 1:10 and preferably from about 1:2.5 to about 1:3. Mixtures of alcohols and paraffins can also be used as the hydrogenation solvent. Preferred mixtures include isoocatane/ethanol, preferably about 80/20 by weight and heptane/methanol, preferably about 70/30 by weight.

The catalyst that can be used in the process according to the invention is any hydrogenation catalyst known to those skilled in the art. Such catalysts include, but are not limited to, platinum, palladium, nickel, rhodium, ruthenium, raney nickel and the like. The preferred catalyst is palladium. Such catalysts are usually used as heterogeneous catalysts, that is, as finely dispersed solids or absorbed on inert supports such as, for example, carbon, aluminum oxide or kieselguhr to increase activity and stability. A catalyst containing about 5% by weight of palladium on carbon is preferably used for the purposes of the invention. In one preferred embodiment of the invention, the catalyst may even be used in moist form. The amount of catalyst is any amount which is necessary to effect the hydrogenation of a sterol to a stanol to produce a product in a desired yield and having the desired purity under the appropriate reaction conditions. Typically, the amount of catalyst will be from about 0.1 to about 10% by weight of sterol and preferably from about 0.2 to about 5% by weight and even more preferably from about 3 to about 5% by weight. The term active catalyst means the amount of metallic catalyst. The term catalyst means the amount of support plus active catalyst. For example, in Example 1, 4 grams of 5% palladium on active carbon are used to reduce 100 g of phytosterol (GENEROL® 122, Henkel KGaA). The amount of catalyst is 4 grams and the amount of active catalyst is 0.2 grams of palladium. The hydrogenation may be carried out at temperatures of from about 20° C. to about 160° C. and is preferably carried out at temperatures of from about 30° C. to about 70° C. The pressure range is generally between about atmospheric pressure and about 250 bar (2.5·10⁷ Pa) and preferably in the range from about 5 bar (5·10⁵ Pa) to about 50 bar (5·10⁶ Pa). The time of reaction can be any time required to bring about the desired reduction and can be readily determined by those skilled in the art. Reaction times are typically from about 1 h to about 7 h. The reaction vessel used is normally a pressure autoclave equipped with an intensive stirrer and a gassing stirrer. The input of energy through the stirrer is typically between about 2 and about 3 kW/m³ with an optimum at 2.2 kW/m³. In one embodiment of the invention, the hydrogenation is carried out continuously.

The hydrogenated sterols (=stanols) may be purified by methods known to those skilled in the art. An example of such a method comprises first filtering off the catalyst, dissolving the filtrate to remove the solvent and subsequently recrystallizing the stanols. The stanols are then dissolved in hydrocarbons or mixtures of hydrocarbons with short-chain alcohols and water. The preferred solvent is i-octane. The ratio by weight of stanol to solvent is generally from about 1:2 to about 1:10 and preferably about 1:4. The stanols may be dissolved at from about 60 to about 100° C. and preferably from about 75 to about 85° C. The crystallization generally takes place at from about −20 to about 40° C. and preferably at from about 5 to about 20° C. In one preferred embodiment, the hydrogenation is carried out in butanol, in which case the subsequent recrystallization may be carried out directly, i.e., without any need for the solvent to be removed by distillation beforehand.

The following Examples are meant to illustrate but not to limit the invention.

### EXAMPLE 1a

About 100 g of phytosterol (GENEROL® 122, Henkel KGaA) were dissolved in 250 g of decanol and 4 g of catalyst (5% palladium on active carbon) were added to the resulting solution. The mixture was hydrogenated under a hydrogen pressure of 5 bar for 7 h at 50° C. The catalyst was then filtered off and the alcohol was distilled off. The iodine value fell from 70 to 15. The OH value remained unchanged at about 130. To remove the impurities formed, the crude product was dissolved in 4 parts of n-heptane at 80° C. The mixture was cooled for 3 h to 20° C. and the stanols were filtered off. The sum total of impurities (components other than stanol and sterol) in the end product was less than 1%.

### EXAMPLE 1b

The procedure was as in Example 1a except that a Pd/active carbon catalyst containing 50% of water was used. The same results as in Example 1a were obtained.

### EXAMPLE 2

About 12.5 kg of phytosterols (GENEROL® 122, Henkel KGaA) were dissolved in 30 kg of decanol and 0.5 kg of catalyst (5% palladium on active carbon) was added to the resulting solution. The mixture was hydrogenated under 25 bar hydrogen pressure for 4 h at 50° C. After the hydrogenation, the catalyst was filtered off and the alcohol was distilled off. The iodine value fell from about 70 to 15. The OH value remained unchanged at about 130. To remove the impurities formed, the crude product was dissolved in 4 parts of n-octane at 80° C. The mixture was cooled for 3 h to 20° C. and the stanols were filtered off. The sum total of impurities (components other than stanol and sterol) in the end product was less than 1%.

### EXAMPLE 3

About 1.7 t of phytosterol (GENEROL® 122, Henkel KGaA) were dissolved in 4.1 t of decanol and 68 kg of catalyst (5% palladium on active carbon) were added to the resulting solution. The mixture was hydrogenated for 4 h at 50° C. under a hydrogen pressure of 20 bar (2·10⁶ Pa). An 8 m³ stirred autoclave with an internal cooler was used as the reactor. The input of energy through the stirrer amounted to 2.2 kW/m³. After the hydrogenation, the catalyst was filtered off and the alcohol was distilled off. Purification by recrystallization was carried out in the same way as in Example 2. The sum total of impurities (components other than stanol and sterol) in the end product was less than 1%.

### EXAMPLE 4

Hydrogenation of sterols was carried out according to the method of the invention in various solvents. The results are as follows:

| | | Ratio by | | | |
|---|---|---|---|---|---|
| | | weight of | Reaction | | |
| | | sterol to | temperature | Iodine | Hydroxyl |
| Solvent | | solvent | [° C.] | value | value |
| a | Butanol | 1:4.4 | 60 | 7.5 | 138.4 |
| b | Isooctane | 1:5 | 80 | 9.7 | 122.5 |
| c | Isooctane/ethanol | 1:5 | 80 | 11.2 | 160.2 |
| | (80/20) | | | | |
| d | Heptane/methanol | 1:5 | 80 | 29.3 | 133.4 |
| | (70/30) | | | | |
| e | Heptane/methanol | 1:5 | 70 | 22.4 | 142.2 |
| | (70/30) | | | | |

What is claimed is:

1. A process for the production of stanols, which comprises contacting a sterol with hydrogen in the presence of a catalyst-effective amount of a hydrogenation catalyst and an organic solvent wherein the ratio by weight of the sterol to the solvent is from about 1:2 to about 1:10.

2. The process of claim 1 wherein the solvent is an alcohol of the formula (I):
R¹—OH
wherein R¹ is an aliphatic, linear or branched hydrocarbon radical having from 1 to about 22 carbon atoms.

3. The process of claim 1 wherein the solvent is an aliphatic, linear or branched paraffinic hydrocarbon having from about 5 to about 16 carbon atoms.

4. The process of claim 1 wherein the solvent comprises
a) an alcohol selected from the group consisting of the alcohols of formula (I):
R¹—OH
wherein R¹ is an aliphatic, linear or branched hydrocarbon radical having from 1 to about 22 carbon atoms and mixtures thereof, and
b) an hydrocarbon selected from the groups consisting of aliphatic, linear or branched paraffin hydrocarbons containing 5 to 16 carbon atoms and mixtures thereof.

5. The process of claim 1 wherein the process is carried out at a temperature of from about 20° C. to 160° C.

6. The process of claim 1 wherein the process is carried out at a pressure of from about atmospheric pressure to about 250 bar hydrogen pressure.

7. The process of claim 1 wherein the sterol is a phytosterol.

8. The process of claim 7 wherein the phytosterol is sitosterol.

9. The process of claim 1 wherein the catalyst is selected from the group consisting of platinum, palladium, nickel, rhodium, ruthenium, raney nickel and combinations thereof.

10. The process of claim 9 wherein the catalyst is palladium.

11. The process of claim 9 wherein the catalyst is absorbed on an inert support.

12. The process of claim 10 wherein the palladium is absorbed on carbon.

13. The process of claim 1 wherein the amount of catalyst is from about 0.01% to about 10% of catalyst by weight of sterol.

14. The process of claim 1 further comprising the step of purifying the stanol by recrystallization.

15. The process of claim 14 wherein the stanol is recrystallized from the same solvent as used in the hydrogenation step.

16. The process of claim 14 wherein the stanol is recrystallized from isooctane.

17. The process of claim 14 wherein the ratio by weight of stanol to solvent in the recrystallization step is from about 1:2 to about 1:10.

18. A process for the production of stanols, which comprises contacting a sterol with hydrogen in the presence of a catalyst-effective amount of a hydrogenation catalyst and an organic solvent wherein the sterol is a phytosterol; wherein the solvent comprises:
a) an alcohol selected from the group consisting of the alcohols of the formula (I):
R¹—OH
wherein R¹ is an aliphatic, linear or branched hydrocarbon radical having from 1 to about 22 carbon atoms and mixtures thereof, and
b) a hydrocarbon selected from the groups consisting of aliphatic, linear or branched paraffin hydrocarbons containing 5 to 16 carbon atoms and mixtures thereof;
wherein the catalyst is palladium; wherein the ratio by weight of the sterol to the solvent is from about 1:2 to about 1:10.

19. The process of claim 18 further comprising the step of purifying the stanol by recrystallization.

20. A process for the production of stanols, which comprises contacting a sterol with hydrogen in the presence of a catalyst-effective amount of a hydrogenation catalyst and an organic solvent wherein the sterol is a phytosterol; wherein the solvent comprises an alcohol selected from the group consisting of the alcohols of the formula (I):
R¹—OH
wherein R¹ is an aliphatic, linear or branched hydrocarbon radical having from 1 to about 22 carbon atoms and mixtures thereof, and
wherein the catalyst is palladium; wherein the ratio by weight of the sterol to the solvent is from about 1:2 to about 1:10.
